(19) United States
(12) Patent Application Publication
(10) Pub. No.: US 2023/0255523 A1
(43) Pub. Date: Aug. 17, 2023

(54) DEVICE FOR MEASURING PERCUTANEOUS OXYGEN SATURATION, AND METHOD FOR CONTROLLING SAME

(71) Applicant: MEDICOSON CO., LTD., Wonju-si (KR)

(72) Inventor: Cheol Hee Won, Wonju-si (KR)

(73) Assignee: MEDICOSON CO., LTD., Wonju-si (KR)

(21) Appl. No.: 18/004,017

(22) PCT Filed: Oct. 15, 2020
(86) PCT No.: PCT/KR2020/014076

§ 371 Date: Feb. 1, 2023

(30) Foreign Application Priority Data
Jun. 30, 2020 (KR) 10-2020-0080266

(51) Int. Cl. A61B 5/1455; A61B 5/00; A61B 5/0205; A61B 5/1491; A61B 5/1495

(52) U.S. Cl. CPC A61B 5/14551; A61B 5/7228; A61B 5/7225; A61B 5/02055; A61B 5/1491; A61B 5/1495; A61B 2562/0271; A61B 2560/0223

(57) ABSTRACT

The present invention relates to a peripheral oxygen saturation measurement device, the device including a light projection unit including a plurality of light sources which project pieces of light in different wavelength bands, a drive unit which drives the plurality of light sources at designated frequencies, a code modulation unit which modulates the frequencies at which the plurality of light sources are driven using at least one designated code, a light receiving unit configured to receive a light signal, which is projected from the light projection unit and passes through a user's blood vessel, convert the received light signal into an electrical signal, and outputs the electrical signal, a signal amplifying unit which amplifies the output signal of the light receiving unit, a code demodulation unit which demodulates the signal amplified by the signal amplifying unit using the same code used in the code modulation unit, and a control unit which calculates peripheral oxygen saturation using the signal projected through the light projection unit and the signal demodulated through the code demodulation unit.

### TECHNICAL FIELD

The present invention relates to a peripheral oxygen saturation measurement device and a method of controlling the same, and more specifically, to a peripheral oxygen saturation measurement device capable of rapidly and accurately measuring peripheral oxygen saturation by performing code spreading modulation on pieces of light in different wavelength bands, simultaneously projecting the pieces of light onto a blood vessel, and performing code dispreading demodulation on pieces of light, which pass through the blood vessel and are simultaneously received, and a method of controlling the same.

### BACKGROUND ART

Generally, peripheral oxygen saturation (SpO₂) is blood oxygen saturation measured by a pulse oximeter mounted on an end of a finger (or earlobe) as illustrated in FIG. 1.

SpO₂[%] is calculated as “hemoglobin (Hb) bound to oxygen/total Hb (that is, Hb bound to oxygen+Hb not bound to oxygen)*100%.”

In other words, Hb in red blood cells may be divided according to whether it is bound to oxygen or not. Hb bound to oxygen is called oxygenated Hb (OxyHb), and Hb not bound to oxygen is called deoxygenated Hb (DeoxyHb). OxyHb and DeoxyHb differ in the degree of absorption of pieces of light (such as, infrared light) in different wavelength bands (that is, absorption coefficients of OxyHb and DeoxyHb are different). Using this principle, pieces of light in different wavelength bands are projected onto a blood vessel to measure peripheral oxygen saturation (SpO₂).

For reference, a longer wavelength means a lower frequency per second, and a shorter wavelength means a higher frequency per second. Therefore, in order to improve signal processing efficiency, a low frequency per second is preferable.

A normal range of an SpO₂ value is 95% or more, a value of 91 to 94% is a value that requires monitoring, and a value of 90% or less is a risk value corresponding to acute respiratory failure and has recently been used as an important indicator when determining a patient who is seriously ill due to the novel coronavirus infection disease (COVID-19). Therefore, the measurement accuracy of a peripheral oxygen saturation (SpO₂) measurement device is very important.

However, since the principle of the peripheral oxygen saturation (SpO₂) measurement device is that the peripheral oxygen saturation (SpO₂) measurement device is mounted on an earlobe or finger to measure the peripheral oxygen saturation and pulse of arterial blood through local perfusion, in a situation in which peripheral blood vessels constrict during measurement (for example, in a situation in which a medicine is injected or a body temperature decreases), local perfusion rapidly deteriorates over time, and thus it is difficult to accurately measure peripheral oxygen saturation. In addition, there is also a problem that measurement accuracy is lowered due to ambient noise input through a sensor.

Therefore, there is a need for a peripheral oxygen saturation (SpO₂) measurement device capable of rapidly and accurately measuring peripheral oxygen saturation (SpO₂) by minimizing the measurement time and an influence of ambient noise to measure peripheral oxygen saturation (SpO₂) and also maintaining reliability.

The related art of the present invention is disclosed in Korean Patent Laid-Open No. 10-2017-0128657 (Published on Nov. 23, 2017. Title of Invention: SpO₂ measuring system and measuring method therewith).

### Technical Problem

According to one aspect of the present invention, the present invention was invented to address the above problem and is directed to providing a peripheral oxygen saturation measurement device capable of rapidly and accurately measuring peripheral oxygen saturation by performing code spreading modulation on pieces of light in different wavelength bands, simultaneously projecting the pieces of light onto a blood vessel, and performing code dispreading demodulation on pieces of light, which are simultaneously received after passing through the blood vessel, and a method of controlling the same.

### Technical Solution

One aspect of the present invention provides a peripheral oxygen saturation measurement device, the device including a light projection unit including a plurality of light sources which project pieces of light in different wavelength bands, a drive unit which drives the plurality of light sources at designated frequencies, a code modulation unit which modulates the frequencies at which the plurality of light sources are driven using at least one designated code, a light receiving unit configured to receive a light signal, which is projected from the light projection unit and passes through a user's blood vessel, convert the received light signal into an electrical signal, and output the electrical signal, a signal amplifying unit which amplifies the output signal of the light receiving unit, a code demodulation unit which demodulates the signal amplified by the signal amplifying unit using the same code used in the code modulation unit, and a control unit which calculates peripheral oxygen saturation using the signal projected through the light projection unit and the signal demodulated through the code demodulation unit.

The control unit may generate the at least one designated code to be used by the code modulation unit and store the least one designated code in an internal memory, and the code demodulation unit may use the least one designated code during signal demodulation.

The device may further include a body temperature detection unit which detects a body temperature at a corresponding portion of the body at which the peripheral oxygen saturation measurement device measures peripheral oxygen saturation and a heating unit which raises the temperature of the corresponding portion of the body to a predetermined standard when the detected body temperature is lower than the predetermined standard, wherein the control unit may reflect a correction value in a measured peripheral oxygen saturation value on the basis of a preset lookup table until the body temperature rises above a predetermined body temperature.

The device may be implemented so that a test jig for checking a state of the device is inserted into the device and further include a jig information detection unit which detects the insertion of the test jig and peripheral oxygen saturation information set in the inserted test jig, wherein the jig information detection unit may include a plurality of terminals for detecting the insertion of the test jig and detecting information of the inserted test jig.

Another aspect of the present invention provides a method of controlling a peripheral oxygen saturation measurement device, the method including generating, by a control unit of the peripheral oxygen saturation measurement device, at least one code for modulating a frequency corresponding to light signals to be simultaneously projected by a light projection unit and storing the code in an internal memory, modulating, by the control unit, the frequency corresponding to the light signals to be simultaneously projected using the at least one generated code, simultaneously driving, by the control unit, light sources of the light projection unit at the modulated frequency and projecting the corresponding light signals through the light projection unit, receiving, by the control unit, the light signals through a light receiving unit and demodulating the received light signals using the same code used in the modulating, and calculating or measuring, by the control unit, peripheral oxygen saturation using the projected light signals and the demodulated light signals.

The method may further include simultaneously measuring, by the control unit, a body temperature when measuring the peripheral oxygen saturation, checking, by the control unit, whether the measured body temperature is greater than or equal to a predetermined body temperature, and, when the measured body temperature is not greater than or equal to the predetermined body temperature, turning, by the control unit, a heating unit on and reflecting a correction value in a measured peripheral oxygen saturation value on the basis of a preset lookup table until the body temperature rises above the predetermined body temperature.

The method may further include, when insertion of a test jig into the peripheral oxygen saturation measurement device is detected, switching, by the control unit, a peripheral oxygen saturation measurement mode to a state check mode and retrieving peripheral oxygen saturation information set in the test jig, projecting, by the control unit, light onto the test jig and measuring an actual peripheral oxygen saturation of the test jig, comparing, by the control unit, the peripheral oxygen saturation actually measured in the test jig with the peripheral oxygen saturation information retrieved from the jig, and outputting, by the control unit, a state of the peripheral oxygen saturation measurement device on the basis of a result of the comparing of the actual peripheral oxygen saturation with the peripheral oxygen saturation set in the test jig and determining whether the actual peripheral oxygen saturation and the peripheral oxygen saturation set in the test jig are similar within an error range.

### Advantageous Effects

According to one aspect of the present invention, the present invention allows peripheral oxygen saturation to be rapidly and accurately measured by performing code spreading modulation pieces of light in different wavelength bands, simultaneously projecting the pieces of light onto a blood vessel, and performing code dispreading demodulation on pieces of light, which are simultaneously received after passing through the blood vessel.

### DESCRIPTION OF DRAWINGS

FIG. 1 is a schematic exemplary view illustrating a conventional peripheral oxygen saturation measurement device.

FIG. 2 is a schematic exemplary view illustrating a configuration of a peripheral oxygen saturation measurement device according to one embodiment of the present invention.

FIG. 3 is a flowchart for describing a method of controlling a peripheral oxygen saturation measurement device according to a first embodiment of the present invention.

FIG. 4 is a flowchart for describing a method of controlling a peripheral oxygen saturation measurement device according to a second embodiment of the present invention.

FIG. 5 is a flowchart for describing a method of controlling a peripheral oxygen saturation measurement device according to a third embodiment of the present invention.

FIG. 6 is an exemplary view for describing a shape of a test jig for the conventional peripheral oxygen saturation measurement device in FIG. 1.

### MODE OF THE INVENTION

Hereafter, embodiments of a peripheral oxygen saturation measurement device and a method of controlling the same according to the present invention will described with reference to the accompanying drawings.

Sizes of components or thicknesses of lines in the drawings may be exaggerated for clarity and convenience of explanation. In addition, some terms described below are defined in consideration of functions in the invention, and meanings may vary depending on, for example, a user or operator's intentions or customs. Therefore, the meanings of terms should be interpreted based on the scope throughout this specification.

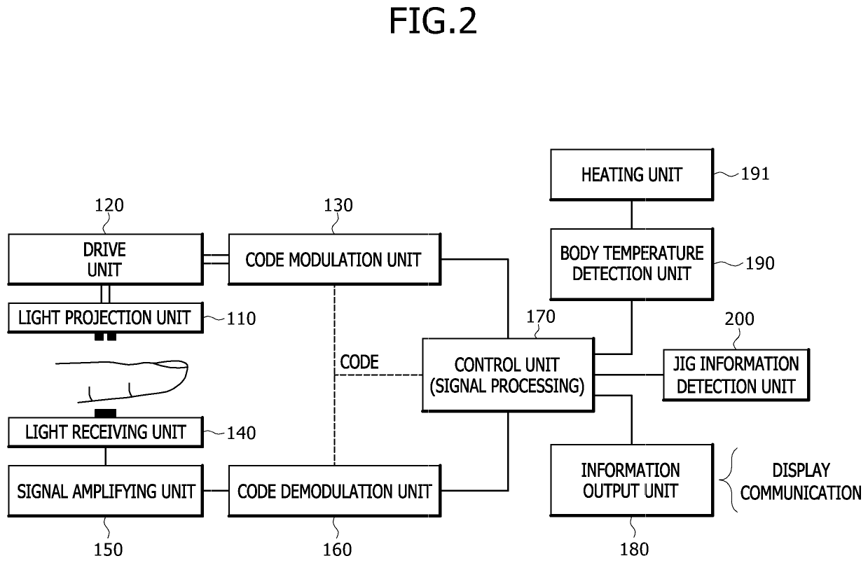

FIG. 2 is a schematic exemplary view illustrating a configuration of a peripheral oxygen saturation measurement device according to one embodiment of the present invention.

As illustrated in FIG. 2, the peripheral oxygen saturation measurement device according to the present embodiment includes a light projection unit 110, a drive unit 120, a code modulation unit 130, a light receiving unit 140, a signal amplifying unit 150, a code demodulation unit 160, a control unit 170, an information output unit 180, a body temperature detection unit 190, a heating unit 191, and a jig information detection unit 200.

The light projection unit 110 includes a plurality of light sources (for example, red light emitting diodes (LEDs) and infrared LEDs) which project pieces of light in different wavelength bands.

In addition, the light projection unit 110 may simultaneously or individually drive the plurality of light sources according to driving of the drive unit 120.

The drive unit 120 may drive the plurality of light sources (for example, the red LEDs and the infrared LEDs) at differently designated frequencies.

In addition, the drive unit 120 may modulate the frequencies for driving the plurality of light sources using a designated code through the code modulation unit 130.

The code modulation unit 130 may perform frequency hopping spread spectrum modulation on a frequency for driving the plurality of light sources using one designated code or perform direct sequence spread spectrum modulation on different frequencies for driving the plurality of light sources using a plurality of designated codes.

In this case, the one designated code or the plurality of designated codes to be used by the code modulation unit 130 may be generated by the control unit 170, and in this case, the generated code (for example, the one code or plurality of codes) may be stored in an internal memory (not shown) and used during signal demodulation by the code demodulation unit 160.

The light receiving unit 140 includes at least one light receiving element (for example, a photodiode) which receives a light signal, which is projected by the light projection unit 110 and passes through a user's blood vessel, converts the received light signal into an electrical signal, and outputs the converted light signal.

The signal amplifying unit 150 amplifies an output signal of the light receiving unit 140.

In this case, the signal amplified and output by the signal amplifying unit 150 may include noise. However, the included noise is removed during a demodulation process using a code designated by the code demodulation unit 160.

The code demodulation unit 160 demodulates a signal amplified by the signal amplifying unit 150 using the same code used by the code modulation unit 130. That is, demodulation is performed using one designated code or a plurality of designated codes according to a method used for modulation by the code modulation unit 130.

The control unit 170 calculates peripheral oxygen saturation using a signal projected through the light projection unit 110 and a signal demodulated through the code demodulation unit 160. In this case, since the principle of calculating peripheral oxygen saturation using a light signal projected through the light projection unit 110 and a light signal received through the light receiving unit 140 is already known, specific description thereof will be omitted.

However, in the present embodiment, since the light signal is modulated using a designated code before being projected, and the modulated signal is demodulated using the same code, noise is removed, and thus even when peripheral oxygen saturation is calculated in the same way as the convention method, there is an effect of improving accuracy as much as the removed noise.

In addition, in the present embodiment, since light signals may be projected by simultaneously driving the plurality of light sources using the light projection unit 110, there is an effect of reducing the peripheral oxygen saturation measurement time as compared to the conventional method of projecting light signals by sequentially driving the plurality of light sources.

The information output unit 180 includes a display (not shown) for outputting a peripheral oxygen saturation value calculated by the control unit 170 and a communication unit (not shown) for transmitting the value to a terminal device (not shown) connected through a cable.

Due to the nature of the peripheral oxygen saturation measurement device which is mounted on an earlobe, the finger, or the like and measures peripheral oxygen saturation, in order to address a problem that accuracy may be lowered when peripheral blood vessels constrict due to a decrease in body temperature during measurement, the body temperature detection unit 190 and the heating unit 191 include a heating unit (for example, a heater) for increasing the body temperature of the finger to a predetermined standard body temperature when the body temperature of a finger is detected and the detected body temperature is lower than the predetermined standard body temperature.

However, since the heating unit 191 is not for raising the body temperature as a whole, but for raising and temporarily maintaining the body temperature at a peripheral oxygen saturation measurement portion to a predetermined standard body temperature, the heating unit 191 is implemented to be operated by a microcurrent.

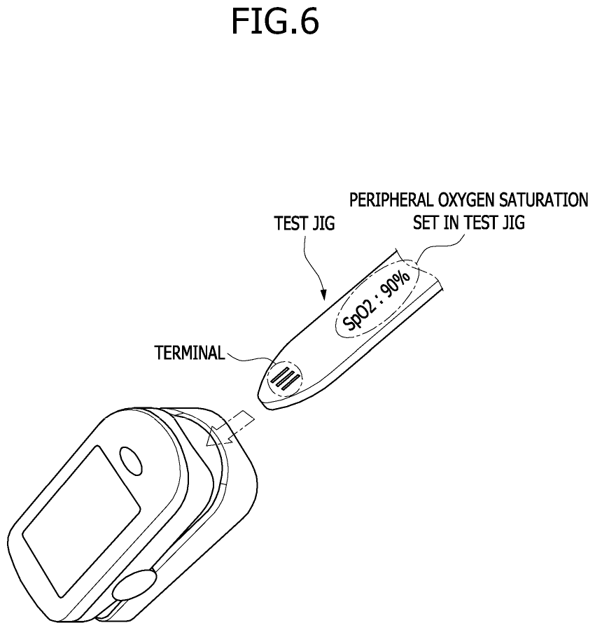

In order to maintain the accuracy of the peripheral oxygen saturation measurement device according to the present embodiment, the jig information detection unit 200 detects the insertion of a jig (that is, a test jig) and information (that is, peripheral oxygen saturation information set in the jig) of the inserted jig upon insertion of the jig, which may frequently check a state (for example, normal or faulty) (see FIG. 6).

In addition, the jig information detection unit 200 may include a plurality of terminals for detecting the insertion of the jig and information (that is, peripheral oxygen saturation information set in the jig) of the inserted jig.

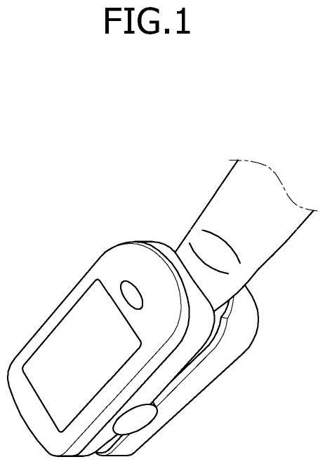

FIG. 6 is an exemplary view for describing a shape of the test jig for the conventional peripheral oxygen saturation measurement device in FIG. 1, and the test jig is implemented in the shape in which the test jig may be inserted into the peripheral oxygen saturation measurement device according to the present embodiment instead of the finger and includes the plurality of terminals, which may be connected to a main body of the peripheral oxygen saturation measurement device according to the present embodiment, at one side.

In this case, driving power may be applied or information stored in the test jig may be transmitted through the plurality of terminals.

In addition, peripheral oxygen saturation information set in the test jig is printed on one side of a surface of the test jig, and the user can visually check the peripheral oxygen saturation information set in the test jig.

In this case, the peripheral oxygen saturation information printed on the surface of the test jig and peripheral oxygen saturation information stored in the test jig are the same.

When the insertion of the jig is detected, the control unit 170 changes a mode to a state check mode (or test mode) of the peripheral oxygen saturation measurement device, compares an actual peripheral oxygen saturation of the jig measured by projecting light onto the jig with the peripheral oxygen saturation value detected by the jig (that is, the peripheral oxygen saturation value set in the jig) to determine whether the actual peripheral oxygen saturation and the peripheral oxygen saturation value (that is, the peripheral oxygen saturation value set in the jig) detected by the jig are similar within an error range. That is, the control unit 170 determines a state as normal when two values (for example, actual peripheral oxygen saturation and peripheral oxygen saturation set in the jig) are similar within an error range, and the control unit 170 determines a state as faulty when the two peripheral oxygen saturation values are not similar within the error range.

For example, the peripheral oxygen saturation measurement device according to the present embodiment has an effect of improving the reliability of the device because the device is used only when a state of the device is first checked and determined as normal using the jig before using the peripheral oxygen saturation measurement device.

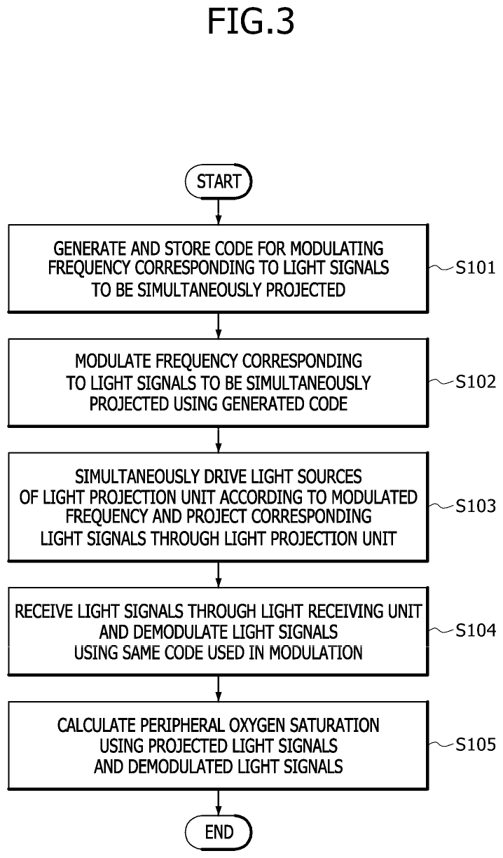

FIG. 3 is a flowchart for describing a method of controlling a peripheral oxygen saturation measurement device according to a first embodiment of the present invention.

Referring to FIG. 3, a control unit 170 generates at least one code to modulate a frequency corresponding to light signals to be simultaneously projected by a light projection unit 110 of the peripheral oxygen saturation measurement device, and stores the code in an internal memory (not shown) (S101).

In addition, the control unit 170 modulates the frequency corresponding to the light signals to be simultaneously projected using the at least one generated code (S102).

In addition, the control unit 170 simultaneously drives light sources of the light projection unit 110 at the modulated frequency and projects the corresponding light signals through the light projection unit (S103).

In addition, the control unit 170 receives light signals through a light receiving unit 140 and demodulates the received light signals using the same code used in the modulation (S104).

In addition, the control unit 170 calculates peripheral oxygen saturation using the projected light signals and the demodulated light signals (S105).

However, in the present embodiment, since the light signals are modulated using the designated code before projecting the light signals, and the modulated signals are demodulated using the same code, noise is removed, and thus there is an effect of improving the measurement accuracy of peripheral oxygen saturation as much as the removed noise. In addition, since the plurality of light sources can be simultaneously driven to project the light signals instead of the conventional method of sequentially driving the plurality of light sources and projecting the light signals, there is an effect of reducing the peripheral oxygen saturation measurement time.

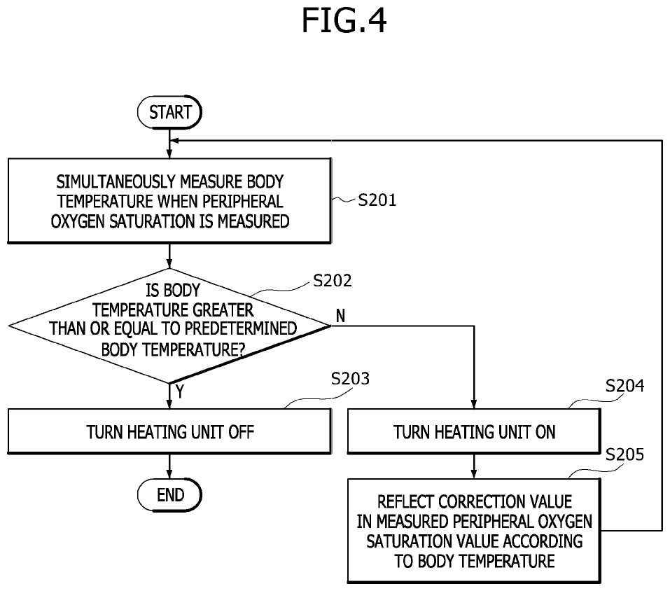

FIG. 4 is a flowchart for describing a method of controlling a peripheral oxygen saturation measurement device according to a second embodiment of the present invention.

Referring to FIG. 4, a control unit 170 simultaneously measures a body temperature (for example, a body temperature of a finger at which peripheral oxygen saturation is measured) when measuring peripheral oxygen saturation using the peripheral oxygen saturation measurement device according to the present embodiment (S201).

In addition, the control unit 170 checks whether the measured body temperature is greater than or equal to a predetermined body temperature (for example, a range of 36.5±0.2 degrees) (S202).

Accordingly, when the measured body temperature is greater than or equal to the predetermined body temperature (for example, the range of 36.5±0.2 degrees) (Y in S202), peripheral oxygen saturation is continuously measured in a state in which a heating unit is turned off (S203).

However, when the measured body temperature is not greater than or equal to the predetermined body temperature (for example, the range of 36.5±0.2 degrees) (N in S202), after the heating unit is turned on (S204), a correction value is reflected in a peripheral oxygen saturation measurement value on the basis of a preset lookup table (not shown, for example, a lookup table calculated through an experiment) until the body temperature rises to the predetermined body temperature (for example, the range of 36.5±0.2 degrees) (S205).

Accordingly, the present embodiment has an effect of improving the accuracy of measuring peripheral oxygen saturation because a problem that accuracy may decrease when peripheral blood vessels constrict due to a decrease in body temperature during measurement is resolved.

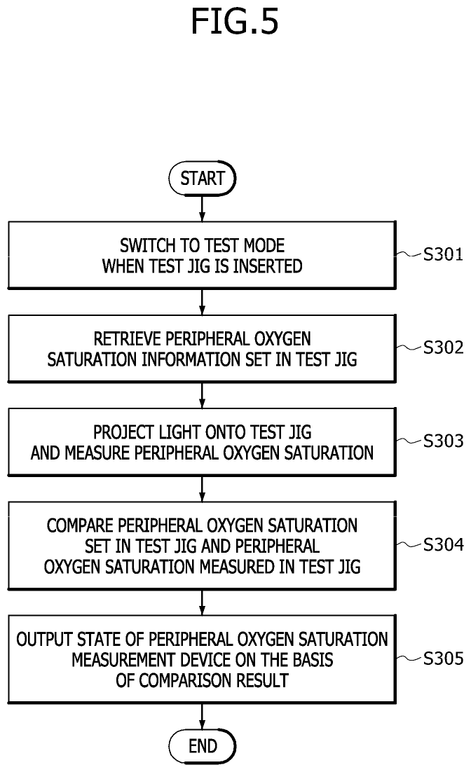

FIG. 5 is a flowchart for describing a method of controlling a peripheral oxygen saturation measurement device according to a third embodiment of the present invention.

Referring to FIG. 5, a control unit 170 switches a peripheral oxygen saturation measurement mode to a state check mode (or test mode) when insertion of a test jig is detected in the peripheral oxygen saturation measurement device according to the present embodiment (S301).

In addition, the control unit 170 retrieves peripheral oxygen saturation information set in the test jig (S302).

In addition, the control unit 170 measures peripheral oxygen saturation by projecting light onto the test jig (S303).

In addition, the control unit 170 compares the peripheral oxygen saturation actually measured using the test jig with the peripheral oxygen saturation information retrieved from the jig (that is, the peripheral oxygen saturation information set in the jig (S304).

In addition, the control unit 170 outputs a state of the peripheral oxygen saturation measurement device on the basis of a result of determining whether two peripheral oxygen saturation values (for example, actual peripheral oxygen saturation and peripheral oxygen saturation set in the jig) are similar within an error range (S305).

For example, when the two peripheral oxygen saturation values are similar within the error range, the control unit 170 determines a state of the peripheral oxygen saturation measurement device as normal, and when the two values of peripheral oxygen saturation are not similar within the error range, the control unit 170 determines a state of the peripheral oxygen saturation measurement device as faulty.

As described above, the present embodiment has an effect of improving the measurement reliability of the peripheral oxygen saturation measurement device because the state of the peripheral oxygen saturation measurement device is determined using the test jig before using the test jig on an actual patient.

While the present invention has been described with reference to embodiments illustrated in the accompanying drawings, this is merely exemplary. It will be understood by those skilled in the art that various modifications and other equivalent example embodiments may be made from the embodiments of the present invention. Therefore, the scope of the present invention is defined by the appended claims. In addition, the present invention described in this specification can be implemented through, for example, a method, a process, an apparatus, a software program, a data stream, or a signal. Even when the present invention is described as being implemented in only a single form (for example, as a method), the described features may be implemented in another form (for example, as an apparatus or program). An apparatus may be implemented using hardware, software, firmware, or the like. A method may be implemented in an apparatus such as a processor which generally refers to a processing device such as a computer, a microprocessor, an integrated circuit, and a programmable logic device. Examples of a processor also include a communication device such as a computer, a cell phone, a portable/personal digital assistant (PDA), and other devices which facilitate information communication between end-users.

1. A peripheral oxygen saturation measurement device comprising:
a light projection unit including a plurality of light sources which project pieces of light in different wavelength bands;
a drive unit which drives the plurality of light sources at designated frequencies;
a code modulation unit which modulates the frequencies at which the plurality of light sources are driven using at least one designated code;
a light receiving unit configured to receive a light signal, which is projected from the light projection unit and passes through a user's blood vessel, convert the received light signal into an electrical signal, and output the electrical signal;
a signal amplifying unit which amplifies the output signal of the light receiving unit;
a code demodulation unit which demodulates the signal amplified by the signal amplifying unit using the same code used in the code modulation unit; and
a control unit which calculates peripheral oxygen saturation using the signal projected through the light projection unit and the signal demodulated through the code demodulation unit.

2. The device of claim 1, wherein:
the control unit generates the at least one designated code to be used by the code modulation unit and stores the least one designated code in an internal memory; and
the code demodulation unit uses the least one designated code during signal demodulation.

3. The device of claim 1, further comprising:
a body temperature detection unit which detects a body temperature at a corresponding portion of a body at which the peripheral oxygen saturation measurement device measures peripheral oxygen saturation; and
a heating unit which raises the temperature of the corresponding portion of the body to a predetermined standard when the detected body temperature is lower than the predetermined standard,
wherein the control unit reflects a correction value in a measured peripheral oxygen saturation value on the basis of a preset lookup table until the body temperature rises above a predetermined body temperature.

4. The device of claim 1, which is implemented so that a test jig for checking a state of the device is inserted into the device, the device further comprising a jig information detection unit which detects the insertion of the test jig and peripheral oxygen saturation information set in the inserted test jig,
wherein the jig information detection unit includes a plurality of terminals for detecting the insertion of the test jig and detecting information of the inserted test jig.

5. A method of controlling a peripheral oxygen saturation measurement device, the method comprising:
generating, by a control unit of the peripheral oxygen saturation measurement device, at least one code for modulating a frequency corresponding to light signals to be simultaneously projected by a light projection unit and storing the code in an internal memory;
modulating, by the control unit, the frequency corresponding to the light signals to be simultaneously projected using the at least one generated code;
simultaneously driving, by the control unit, light sources of the light projection unit at the modulated frequency and projecting the corresponding light signals through the light projection unit;
receiving, by the control unit, the light signals through a light receiving unit and demodulating the received light signals using the same code used in the modulating; and
calculating or measuring, by the control unit, peripheral oxygen saturation using the projected light signals and the demodulated light signals.

6. The method of claim 5, further comprising:
simultaneously measuring, by the control unit, a body temperature when measuring peripheral oxygen saturation;
checking, by the control unit, whether the measured body temperature is greater than or equal to a predetermined body temperature; and
when the measured body temperature is not greater than or equal to the predetermined body temperature, turning, by the control unit, a heating unit on and reflecting a correction value in a measured peripheral oxygen saturation value on the basis of a preset lookup table until the body temperature rises above the predetermined body temperature.

7. The method of claim 5, further comprising, when insertion of a test jig into the peripheral oxygen saturation measurement device is detected:
switching, by the control unit, a peripheral oxygen saturation measurement mode to a state check mode and retrieving peripheral oxygen saturation information set in the test jig;
projecting, by the control unit, light onto the test jig and measuring an actual peripheral oxygen saturation of the test jig;
comparing, by the control unit, the peripheral oxygen saturation actually measured in the test jig with the peripheral oxygen saturation information retrieved from the jig; and
outputting, by the control unit, a state of the peripheral oxygen saturation measurement device on the basis of a result of the comparing of the actual peripheral oxygen saturation with the peripheral oxygen saturation set in the test jig and determining whether the actual peripheral oxygen saturation and the peripheral oxygen saturation set in the test jig are similar within an error range.
